(19) United States
(12) Patent Application Publication
(10) Pub. No.: US 2008/0193416 A1
(43) Pub. Date: Aug. 14, 2008

(54) Herpes Virus-Based Compositions and Methods of Use

(75) Inventors: Howard J. Federoff, Bethesda, MD (US); William J. Bowers, Webster, NY (US)

Correspondence Address: FISH & RICHARDSON P.C., P.O BOX 1022, Minneapolis, MN, 55440-1022, US

(73) Assignee: University of Rochester, Rochester, NY (US)

(21) Appl. No.: 11/916,440

(22) PCT Filed: Jun. 5, 2006
(86) PCT No.: PCT/US06/21824

§ 371 Date: Mar. 28, 2008

Related U.S. Application Data
(60) Provisional application No. 60/687,360, filed on Jun. 3, 2005.

(51) Int. Cl. A61K 48/00; C12N 15/63; C07H 21/04; C12N 5/06; C12N 15/11; C12Q 1/48; C12Q 1/37; C12Q 1/26; C12Q 1/02; C40B 40/02; A61K 31/7052; C12N 7/00

(52) U.S. Cl. 424/93.2; 435/320.1; 536/23.1; 514/44; 435/235.1; 435/325; 506/14; 435/455; 435/461; 435/29; 435/15; 435/25; 435/23

(57) ABSTRACT

This disclosure features vectors, targeting vectors, modified artificial chromosomes, herpes virus particles in which such chromosomes are packaged, and methods of using the herpes virus particles to identify therapeutic agents and targets for therapeutic intervention.

### RELATED APPLICATIONS

This application claims the benefit of an earlier-filed provisional application, U.S. Ser. No. 60/687,360, filed Jun. 3, 2005, the content of which is hereby incorporated by reference in its entirety.

### STATEMENT REGARDING GOVERNMENT SUPPORT

The work described herein was funded, in part, by grants from the National Institutes of Health. The United States government may, therefore, have certain rights in the invention.

### FIELD OF THE INVENTION

The present invention relates generally to vectors, targeting vectors, modified artificial chromosomes, herpes virus particles in which such chromosomes are packaged, and methods of using the herpes virus particles to identify therapeutic agents and targets for therapeutic intervention.

### SUMMARY

The present invention is based, in part, on our discovery that targeting vectors containing certain elements of herpes viruses can be used to generate modified artificial chromosomes. These chromosomes, which can include a transgene, can be packaged into herpes virus particles, and the particles can be used for functional genomic studies and in therapeutics. Herpes viruses are highly versatile gene delivery platforms. The virions have the capacity to carry large transgenes. For example, the transgene can include up to about 150 kb or more (e.g., 50, 60, 70, 75, 80, 85, 90, 95, 100, 125, 150, or 175 kb or more). The virions are also typically able to infect a variety of types of cells, regardless of whether or not the cell is dividing. Thus, the virions can be used to deliver genetic material to neurons and other post-mitotic cells. The particles also exhibit low, and can exhibit essentially no, toxicity because they lack viral genes.

Accordingly, the compositions of the invention include targeting vectors and modified artificial chromosomes, which can be made from existing artificial chromosomes or generated de novo. The invention also features altered herpes viruses that have packaged the modified artificial chromosomes and compositions that contain them (e.g., pharmaceutical formulations and cells that include the modified artificial chromosomes and/or herpes viruses that contain them). The compositions of the invention can be configured as arrays. For example, cells containing the modified artificial chromosomes and/or the altered herpes viruses can be arrayed (e.g., in the wells of multi-well tissue culture plates or other compartmentalized devices). Cells can be arrayed and then transduced with the modified herpes viruses or transduced and then arrayed. Such arrays are within the scope of the present invention, and they can be used in screening assays aimed at identifying targets for therapeutic intervention (e.g., a cell-surface receptor, second messenger, enzyme, or transcription factor within a biochemical pathway).

While cells within an array can be useful for, for example, high-throughput screening, cells in other configurations (e.g., homogeneous or heterogeneous populations of cells in tissue or organ cultures or in vivo) can also be transduced with altered herpes viruses. While the precise configuration of the assay can vary, following transduction, one can determine whether a given transgene encodes a protein that affects a therapeutic target (thereby identifying the therapeutic target) and/or whether the transgene itself encodes a therapeutic protein. Where the transgene encodes a therapeutic protein, that transgene can subsequently be delivered, using the altered herpes viruses of the invention or any other delivery vehicle, to a patient in need of treatment. Thus, methods of identifying therapeutic targets and/or therapeutic agents, compositions containing those agents, and methods of administering them (e.g., to a cell (e.g., a cell in vivo)) are within the scope of the present invention. The therapeutic target may be a primary target, which is directly affected by the transgene product (e.g., a receptor is a primary target where the transgene product binds and alters (e.g., inhibits) the receptor's activity). The therapeutic target can also be a secondary target, which is one that operates in the same biochemical pathway as the primary target. For example, if a transgene product binds and inhibits a receptor's activity in a therapeutically beneficial way, one could then readily design therapeutic agents that inhibit one or more of the proteins that are active in the signal transduction pathway between the receptor and the effector (i.e., one or more of the secondary targets). Once a target has been identified, one can make and use therapeutic agents other than those encoded by the transgene. For example, where a therapeutically effective transgene encodes a receptor antagonist, one can, if desired, use receptor antagonists other than the one encoded by the transgene. For example, one could use a ligand engineered to bind the receptor but inhibit signal transduction or an antibody that specifically binds and inhibits the receptor. Other agents that inhibit the target by inhibiting its expression can also be administered (e.g., antisense oligonucleotides or siRNAs or other molecules that mediate RNAi). Similarly, where a therapeutically effective transgene encodes an enzyme, such as a phosphatase or kinase, one can administer the transgene to treat a patient or another agent that achieves the same result. For example, one can administer an expression construct that encodes the enzyme or a biologically active variant or fragment thereof.

The therapeutic agents can be delivered in combination with one another and/or in combination with presently known therapeutic agents.

We use the term “protein(s)” to refer to polymers of naturally or non-naturally occurring amino acid residues, whether glycosylated or not, and whether otherwise post-translationally modified or not. We may also refer to these polymers as “polypeptides” or “peptides”.

While the screening methods of the invention are described further below, we note here that a library of altered herpes viruses that express various nucleic acid sequences (e.g., genomic or cDNA sequences from a human or a pathogen) can be used to identify genes important for a variety of physiological events (e.g., cell division, signal transduction, hormone production and secretion, motility, differentiation, muscle contraction, energy production, metabolism, neuroprotection or neuroregeneration). Using the methods described here, one can retrofit any library of existing artificial chromosomes so they can be converted into, or packaged within, herpes virus virions. Alternatively, one can generate new libraries of artificial chromosomes that can be packaged by virions. The retrofit includes inserting, preferably into each clone within the library (e.g., a BAC library), a cleavage/packaging signal (also known as an a sequence/segment or pac) and an ori (the origin of replication, also referred to as a c region) from a herpes virus. Generating new libraries requires providing parental vectors that include the a sequence and an ori, and using those vectors to generate a library of artificial chromosomes. Once the transgenes from the artificial chromosomes are packaged in the virions, cells can be transduced with the virions and examined to determine whether the transgene affects or alters a cellular process (e.g., cell survival, the rate of cell division, cell fate, regenerative ability, or any of the other cellular processes referred to herein).

Other features and advantages of the invention will be apparent from the drawings, the following detailed description, and from the claims.

### BRIEF DESCRIPTION OF THE DRAWINGS

FIG. 1 is a schematic representation of a method that can be used to generate a modified artificial chromosome.

FIG. 2 is a schematic representation of an FRT site. The sequence of the FRT site is composed of three 13-bp symmetry elements (horizontal elements labeled a, b, and c) surrounding an asymmetrical 8-bp core (open box). FLP-mediated cleavage sites are indicated by two small vertical arrows.

FIG. 3 is a Table of essential HSV-1 genes.

FIGS. 4A and 4B are schematic representations of the HSV-1 genome and the overlapping set of five cosmids C6Δa48Δa (cos 6Δa, cos 28, cos 14, cos 56, and cos 48Δa; Fraefel et al., J. Virol. 70:7190-7197, 1996). In the HSV-1 genome of FIG. 5A, only the IE4 gene, oriS and oriL are shown. The a sequences, which contain the cleavage/packaging sites, are located at the junction between the long and short segments and at both termini. In FIG. 5B, the deleted a sequences in cos 6Δa and cos 48Δa are indicated by “X”.

### DETAILED DESCRIPTION

We begin with a description of the targeting vectors and other compositions of matter that can be variously used to practice the methods of the invention.

Targeting vectors and precursors thereof. The vectors we describe as targeting vectors can be made from nucleic acids and, in form, may be linear or circular. For example, the targeting vectors can be plasmids (single- or double-stranded, circularized DNA or RNA molecules). A circularized vector such as a plasmid can be converted to a linear vector by cleaving it at one or more locations. For example, a plasmid can be cleaved at one or more restriction sites or cleavage sites. Alternatively, a linear targeting vector can be made by methods known in the art. For example, one can synthesize and anneal sense and antisense strands of DNA or RNA.

With respect to content, the nucleic acid sequences within the targeting vectors can include a packaging/cleavage site of a herpes virus and an ori of a herpes virus. The packaging/cleavage signal can be any sequence that directs the vector into a particle that is capable of adsorbing to a cell (the cell being the target for transformation). Where the targeting vectors are linear and intended for insertion into a unique or particular site within an artificial chromosome, it is unlikely that any other elements need be present in the targeting vector. Where the targeting vectors participate in reactions where unwanted constructs may form, however, it is beneficial to include additional elements within the targeting vectors that facilitate selection or detection of the modified artificial chromosomes. For example, the ability to discern among possible recombinants can be facilitated by the use of a selectable marker carried with the targeting vector. Accordingly, in addition to the packaging/cleavage site and the ori, a targeting vector can include a sequence that encodes a selectable marker (e.g., an antibiotic resistance gene) and/or a sequence that encodes a detectable marker (e.g., a fluorescent protein). Additional elements may also be present, as may sequences that constitute the backbone of the vector.

The packaging/cleavage site can be that of any herpes virus or a biologically active fragment or other mutant thereof that retains sufficient biological activity to remain useful in the methods of the invention (e.g., a fragment or mutant that retains the ability to package a vector, of which it is a part, into a herpes virus). Similarly, the ori can be that of any herpes virus or an active fragment or other mutant thereof (e.g., a fragment or mutant that retains the ability to mediate replication of nucleic acid sequences).

In specific embodiments, the packaging/cleavage site can be that of HSV-1. Other sequences, including packaging/cleavage sites of other herpesviruses, can be found in the literature or in publicly available databases such as GenBank™. The ori can also be that of an HSV-1. More generally, in any of the compositions described herein that include a packaging/cleavage site and an ori, these elements can be, independently, those of any of the more than 100 known species of herpes virus. For example, the cleavage/packaging site and the ori can be those of an alpha herpes virus (e.g., a Varicella-Zoster virus, a pseudorabies virus, or a herpes simplex virus (e.g., type 1 or type 2 HSV) or an Epstein-Barr virus). The herpes virus can also be a cytomegalovirus. The herpes virus can be a human herpes virus.

Where an HSV element is employed, it can be that of a type 1 (HSV 1) or type 2 (HSV 2) HSV. It can also be that of a type 3 (HSV 3), type 4 (HSV 4), type 5 (HSV 5), type 6 (HSV 6), type 7 (HSV 7), or type 8 (HSV 8) herpes simplex virus. The cleavage/packaging site and the ori can also be those of a human herpes virus. In specific embodiments, the cleavage/packaging site and the ori can be those of HSV 1, and a modified artificial chromosome that incorporates them can be packaged in an HSV 1 virion. In other embodiments, the cleavage/packaging site, the ori, and the virus can be HSV 2; and so on.

The selectable marker can be any protein that facilitates separation of the cells that express the marker from the cells that do not. For example, the targeting vector can include a sequence that confers resistance to an antibiotic; cells that express the marker will survive in the presence of the antibiotic, whereas cells that do not express the marker will perish. More specifically, the targeting vectors of the invention can include a sequence encoding a protein that confers resistance to aminopterin, ampicillin, chloramphenicol, erythromycin, kanamycin, hygromycin, spectinomycin, tetracycline, or another antibiotic. The marker may also be a protein that, when expressed, allows a cell to survive in an altered environment. For example, the protein may be a stress protein (e.g., a heat shock protein) that allows a cell to survive in, for example, an environment where the temperature is raised above a normal physiological temperature (e.g., about 37° C.). The targeting vector can include sequences that encode more than one (e.g., two or three) selectable marker, and the advantage of including more than one marker is described further below.

The detectable marker can be essentially any protein; all that is required is that the protein be useful in identifying a cell in which it is expressed. For example, the targeting vectors can include a sequence encoding a protein that is specifically bound by an antibody or other reagent (e.g., a labeled binding partner). The markers may also be detectable by virtue of chemiluminesence or fluorescence. For example, the detectable marker can be a fluorescent protein (e.g., a protein that, upon proper illumination, fluoresces green (e.g., GFP or enhanced GFP (EGFP)), red (e.g., DSred II), or blue). The sequence encoding the detectable marker can be operably linked to a promoter that directs its expression. For example, the promoter can be constitutively active in mammalian cells or cell type-specific. Many such promoters are known and used by those of ordinary skill in the art. As is true for other elements within the targeting vector, the sequence encoding the detectable marker can be incorporated into the modified artificial chromosomes and the virions that package them. For example, the sequence(s) encoding the detectable marker(s) can be flanked by the cleavage sites and recombined with the cleavage/packaging site, the ori, and the sequence encoding the selectable marker into an artificial chromosome.

The elements described above (e.g., the herpes virus cleavage/packaging site, the ori, and the sequences encoding the selectable and/or detectable markers) can be flanked by a pair of cleavage sites, which may constitute any sequences that allow for recombination. For example, the cleavage sites can be a pair of LoxP elements, which can reform following cleavage with Cre recombinase, or a pair of Flp recombination targets (FRTs), which can reform following cleavage with Flp recombinase. Each member of the pair of LoxP elements can have, or can include, the sequence 5′-ataacttcgtataatgtatgctatacgaagttat-3′ (SEQ ID NO: 1). The minimal sequence of the FRT site is believed to include a 34-basepair sequence containing two 13-basepair imperfect inverted repeats separated by an 8-basepair spacer that includes an Xba I restriction site. An additional 13-basepair repeat is found in most FRT sites, but it may not be required for cleavage. Each member of the pair of FRTs can have, or can include, the sequence 5′-gaagttcctattctctagaaagtataggaacttc-3′ (SEQ ID NO:2) or a biologically active fragment or mutant thereof. The FRT site serves as a binding site for Flp recombinase (see, e.g., Gronostajski and Sadowski, Mol. Cell. Biol. 5:3274-3279, 1985; Gronostajski and Sadowski, J. Biol. Chem. 260:12320-12327, 1985; and Gronostajski and Sadowski, J. Biol. Chem. 260:12328-12335, 1985). See also, FIG. 2.

As is true for all of the sequences useful in the present invention, the sequences of the cleavage sites can differ from naturally occurring sequences, from the recited SEQ ID NOs, or from elements within commercially available vectors so long as they retain sufficient activity to be useful in the methods of the present invention. For example, the LoxP element can be a fragment or other mutant that can still be recognized and cleaved by Cre recombinase. We may describe such fragments and other mutants of specified sequences as having biological activity or as being biologically active. The “cleavage site(s)/sequence(s)” are distinct from the “packaging/cleavage site/sequence.”

Biologically active fragments or mutant sequences can be degenerate variants of a naturally occurring or commercially available sequence. Where the nucleic acid sequences within, for example, the targeting vector or a modified artificial chromosome, encode a protein, at least some of the nucleotides in the third position of the codon can vary but yet encode the same amino acid residue. Biologically active fragments or mutant sequences can also be described as substitution, deletion, or addition mutants, where one or more nucleotides (e.g., 1, 2, 3, 4, 5, or more) are substituted, deleted, or added, respectively. Where the nucleic acid sequence encodes a protein, the biologically active nucleic acid sequence can be altered in such a way that the encoded protein contains a different amino acid residue (e.g., a residue that constitutes a conservative substitution), an additional amino acid residue, or fewer amino acid residues. The degree of difference between two sequences or the extent of mutation can also be expressed in terms of a percentage of identity or homology. For example, biologically active fragments or other mutant sequences useful in the present invention can be at least or about 50% (e.g., at least or about 60%, 65%, 70%, 75%, 80%, 85%, 90%, 95%, 96%, 97%, 98% or 99%) identical to or homologous to a corresponding wildtype sequence. More specifically, a biologically active packaging/cleavage site of a herpesvirus (or an ori, an antibiotic resistance gene, a detectable marker, a cleavage site or any other component of the present targeting vectors) can differ from a corresponding packaging/cleavage site of the same type of herpesvirus (e.g., HSV-1) (or an ori, an antibiotic resistance gene, a detectable marker, a cleavage site or any other component of the present targeting vectors) by virtue of deletion, addition, or substitution of one or more amino acid residues and can exhibit the degree of identity or homology described above (e.g., about or at least 90% identical or homologous).

In specific embodiments, the targeting vectors of the invention include at least one (e.g., one, two or three) pair of cleavage sites, one or more cis elements from a herpes virus (e.g., a packaging/cleavage site and/or an ori), a sequence encoding a selectable marker (e.g., an antibiotic resistance gene) and, optionally, a sequence encoding a detectable marker (e.g., a detectable label or tag). A pair of cleavage sites can flank either all or various cis elements and the sequences encoding the selectable and detectable markers. For example, in one embodiment, the targeting vector includes a single pair of cleavage sites that flank a packaging/cleavage site of a herpes virus, an ori of a herpes virus, and an antibiotic resistance gene (e.g., a kanamycin resistance gene (Kan^{r})).

As noted above, the targeting vector can include a second selectable marker that may not lie between a pair of cleavage sites. For example, where the pair of cleavage sites flank a cleavage/packaging site, an ori, and a first resistance gene (e.g., Kan^{r}), the targeting vector may also contain, “outside” the cleavage sites, a second resistance gene (i.e., a gene that confers resistance to an antibiotic other than that to which the first resistance gene is directed). For example, where the first selectable marker is a Kan^{r} sequence, the second selectable marker can be a nucleic acid sequence that confers resistance to aminopterin, ampicillin, chloramphenicol, erythromycin, hygromycin, spectinomycin, or tetracycline. The first antibiotic resistance gene can also be aminopterin, ampicillin, chloramphenicol, erythromycin, hygromycin, spectinomycin or tetracycline resistance gene.

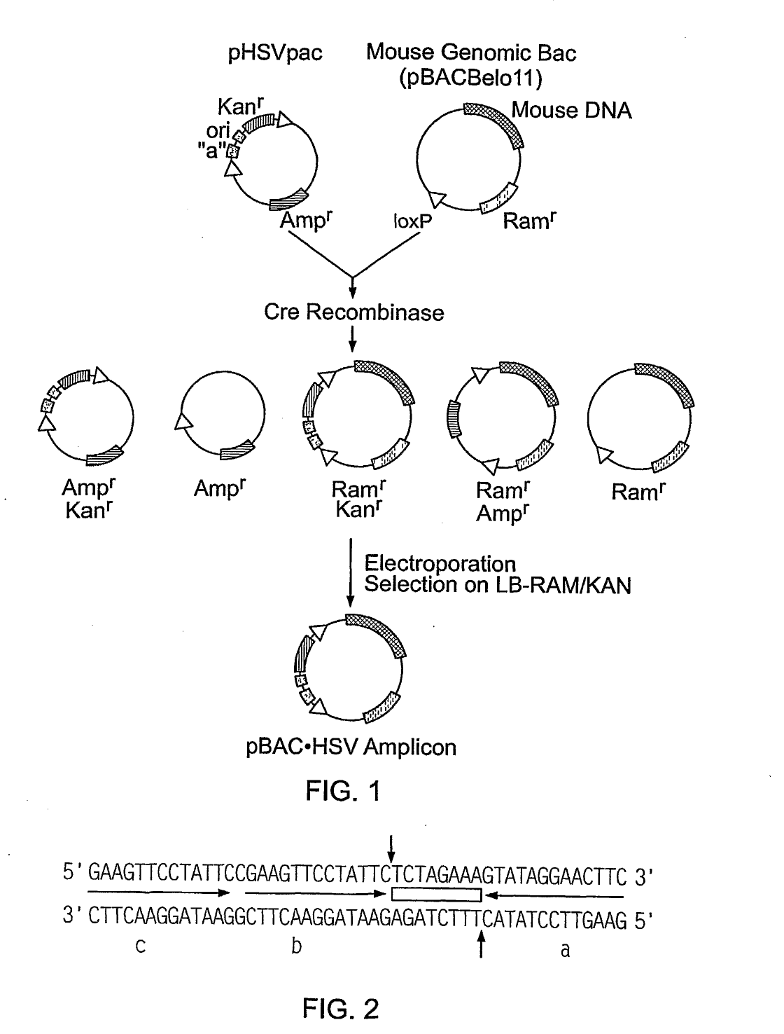

The sequence encoding the second selectable marker may have been present in a vector (e.g., a plasmid (e.g., pBluescript)) used to generate the targeting vector, and certain parental vectors are within the scope of the present invention. For example, the invention features precursor vectors in which either or both of a herpes virus cleavage/packaging site and a herpes virus ori are flanked by unique restriction sites or by a pair of cleavage sites. FIG. 1 is a schematic representation of a method that can be used to generate a modified artificial chromosome. The targeting vector and resulting modified artificial chromosomes, including the pBAC•HSV amplicon and modified artificial chromosomes having the elements of that construct, are within the scope of the present invention.

Using targeting vectors to retrofit an artificial chromosome: Targeting vectors can be used to modify or “retrofit” an artificial chromosome (or a collection thereof) with the a and ori sequences (i.e., to incorporate the a and ori sequences into the artificial chromosome). These two elements are sufficient to confer onto any vector, including the modified artificial chromosomes described herein, the ability to be replicated, cleaved, and inserted into a virion (e.g., an HSV virion). Methods of generating modified artificial chromosomes are described further below. The methods can be carried out by introducing a targeting vector and an artificial chromosome into a cell (e.g., an E. coli strain EL250 containing defective lambda prophage). Those methods, along with methods of inserting the modified chromosomes into virions and using those virions in screening assays and pharmaceutical compositions, are within the scope of the present invention.

We use the term “artificial chromosome” broadly to refer to any non-naturally occurring construct that is capable of incorporating (e.g., into its polymeric structure) large nucleic acid sequences (e.g., sequences greater than about 50 kb). For example, the artificial chromosomes used in the methods of the invention can be yeast artificial chromosomes (YACs), bacterial artificial chromosomes (BACs), and/or human artificial chromosomes (HACs). Within the confines of the upper length limit, these constructs can incorporate essentially any nucleic acid sequence of interest. For example, the constructs can include genomic DNA or cDNA from yeast, bacteria or other pathogens (e.g., viruses, parasites, and fungi), plants (including herbs and particularly including any plant considered to have medicinal properties), or animals. For example, the sequence of interest can be an avian sequence (e.g., a sequence that naturally occurs in a chicken, goose, duck, pheasant, or other bird or a sequence derived therefrom (e.g., a fragment or mutant of an avian sequence)), a reptilian sequence (e.g., a sequence that naturally occurs in a lizard or snake or a sequence derived therefrom (e.g., a fragment or mutant of a reptilian sequence)), an amphibian sequence (e.g., a sequence that naturally occurs in a frog or newt or a sequence derived therefrom (e.g., a fragment or mutant of an amphibian sequence)), or a mammalian sequence (e.g., a sequence that naturally occurs in a sheep, goat, cow, horse, dog, cat, rabbit, pig, human, or rodent (e.g., a rat, mouse, hamster, or guinea pig) or a sequence derived therefrom (e.g., a fragment or other mutant of a mammalian sequence)). Other useful sequences are those of insects (e.g., arthropods), including flies used in research (e.g., D. melanogaster) and other invertebrates (e.g., C. elegans). We may also refer to a nucleic acid sequence of interest as a “transgene.” While artificial chromosomes have the capacity to carry large transgenes, the methods of the invention can be practiced using transgenes of any length.

The artificial chromosomes and modified artificial chromosomes can include more than one transgene that, when expressed, would produce more than one protein or type of protein. For example, the nucleic acid of interest can include several (e.g., 1-5) transgenes that encode several (e.g., 1-5) proteins. For example, the nucleic acid can include transgenes that encode one or more enzymes, receptors, transcription factors, cofactors, extracellular matrix proteins, structural proteins, or other cellular proteins, and the proteins or types of proteins can be the same or different. For example, the nucleic acid of interest can include two transgenes that encode two enzymes, or an enzyme and a structural protein. A given transgene can also be one that encodes an antibody chain or any one of the proteins described herein (see, e.g., the various types and species described above). In the event the nucleic acid of interest within the artificial chromosome or modified artificial chromosome includes more than one transgene, and that nucleic acid produces a desirable effect on a cell, tissue, organ, or animal into which it is introduced (e.g., by way of the modified herpes viruses described herein), one can then isolate and test individual transgenes. For example, one can reduce the size of the nucleic acid (by, for example, exposing it to an endonuclease) so that it encodes only one functional protein or a biologically active fragment thereof. Where one wishes to express a given transgene in a cell, the modified artificial chromosome can be modified to include multiple copies of a transgene.

While it should be clear from the context, we have endeavored to use the term “artificial chromosome” to refer to an artificial chromosome that has not been exposed to, or recombined with elements from, a targeting vector, and the term “modified artificial chromosome” to refer to an artificial chromosome that has been altered to contain desired elements of a targeting vector.

The artificial chromosome can include a sequence encoding a detectable marker, as described herein, e.g., a fluorescent protein, e.g., a green fluorescent protein (for example, enhanced green fluorescent protein EGFP), a red fluorescent protein (for example, DsRedII), or a blue fluorescent protein.

The artificial chromosomes can also include a sequence encoding a selectable marker, which may differ from the selectable marker encoded by the targeting vector. The selectable marker in the artificial chromosome can confer resistance to an antibiotic, including aminopterin, ampicillin, chloramphenicol, erythromycin, kanamycin, hygromycin, spectinomycin, tetracycline, or another antibiotic. For example, the targeting vector can include a sequence encoding a protein that confers resistance to kanamycin, and the artificial chromosome can include a sequence encoding a protein that confers resistance to an antibiotic other than kanamycin (e.g., ampicillin, erythromycin, or tetracycline). When the modified artificial chromosome is generated, it can, then, include two selectable markers. For example, a sequence that confers resistance to neomycin, which can be useful in selecting successfully transduced mammalian cells, and a sequence that confers resistance to ampicillin, which can be useful in selecting successfully transduced bacterial cells (e.g., E. coli).

To generate a modified artificial chromosome, the targeting vector is combined with an artificial chromosome. The artificial chromosomes can contain, as noted above, a sequence of interest and, optionally, a sequence encoding a selectable marker that is distinct from any or all of the selectable markers encoded by the targeting vector. To facilitate recombination, the artificial chromosome can also include at least one cleavage site that is the same as at least one of the cleavage sites in the targeting vector. For example, where the targeting vector includes a pair of LoxP elements, the artificial chromosome can also include a LoxP element. When present, each member of the pair of loxP elements can have or include the sequence 5′-ataacttcgtataatgtatgctatacgaagttat-3′(SEQ ID NO:1) or a biologically active fragment or mutant thereof. Such targeting vectors and artificial chromosomes can be combined in the presence of Cre recombinase under conditions, and for a time, sufficient to allow the Cre recombinase to cleave the LoxP elements in the targeting vector and the artificial chromosome. Upon recombination, at least some of the reaction products will be configured so that the elements previously flanked by the LoxP sites in the targeting vector will be linked to the sequence of interest (or transgene) and, if present, the sequence encoding the selectable marker gene originally present in the artificial chromosome. These reaction products are within the scope of the present invention, as are pure or substantially pure populations of the desired reaction products.

The desired reaction products can be identified and isolated from other reaction products by transfecting cells (e.g., bacterial cells) with the pool of available reaction products, including the desired construct and those that have recombined in ways that are not useful. The cells can then be grown in the presence of antibiotics, which are chosen in view of the selectable marker genes incorporated in the targeting vector and artificial chromosomes. Where a reaction product includes sequences that confer resistance to the antibiotics, the bacterial cell will survive exposure to the antibiotics. For example, where the cleavage elements of the targeting vector flank Kan^{r} and the artificial chromosome includes Ram^{r}, cells that include modified artificial chromosomes that have recombined in a useful way, and therefore contain both of those resistance genes, will grow on (or in) culture medium containing kanamycin and chloramphenicol.

Targeting vectors that include other cleavage sites can be used to generate modified artificial chromosomes in an analogous way. For example, where the targeting vector includes a pair of FRTs, the artificial chromosome can also include one or more FRTs. Such targeting vectors and artificial chromosomes can be combined in the presence of Flp recombinase under conditions, and for a time, sufficient to allow the Flp recombinase to cleave the FRTs in the targeting vector and the artificial chromosome. Subsequently, the sequence between the FRTs in the targeting vector can be recombined with the FRT-cleaved artificial chromosome.

Targeting vectors and artificial chromosomes that include unique sequences recognized by a restriction endonuclease can also be recombined. For example, the a site and ori can be flanked by sequences that are recognized and cleaved by a restriction endonuclease that does not recognize or cleave the targeting vector at any other site. The modified chromosome can include the same sequence. The digested targeting vectors and artificial chromosomes can then be incubated together in the presence of a ligase. As with any genetic engineering, where the restriction endonuclease generates overhanging (as opposed to blunt) ends, the recombination is likely to be more efficient.

Linear targeting vectors: Instead of a circularized targeting vector, such as a plasmid, one can use a linear targeting vector, which we may also refer to herein as a “cassette”. Thus, the invention encompasses linear, double-stranded targeting vectors that include a cleavage/packaging site, an ori and, optionally, sequences encoding a selectable marker and/or a sequence encoding a detectable marker. The linear cassette can be recombined with an artificial chromosome (or a portion thereof) to generate a modified artificial chromosome. The ends of the linear cassette can be blunt or, to better facilitate recombination, the ends of the sense and antisense strands within the cassette can be staggered and complementary to cleavage sites generated within the artificial chromosome.

The methods employing linear cassettes are similar to those that employ circular targeting vectors; the linear cassette and a linearized artificial chromosome (or a portion thereof (e.g., a portion including a sequence of interest and a selectable marker gene)) are combined under conditions, and for a time, sufficient to allow recombination and the formation of a modified artificial chromosome. Selection can be carried out by transfecting cells (e.g., E. coli) with the resultant constructs, some of which will be properly recombined artificial chromosomes, and culturing the cells in the presence of antibiotics. For example, where the linearized targeting vector includes a sequence that confers resistance to ampicillin and the artificial chromosome (or the portion thereof) includes a sequence that confers resistance to tetracycline, properly modified artificial chromosomes can be selected on the basis of their ability to confer, to cells that contain them, resistance to ampicillin and tetracycline.

The modified artificial chromosomes generated using linear targeting vectors can be packaged in the herpes viruses described herein and used in the screening assays and therapeutic regimes described below ((just as if they had been generated using a non-linear targeting vector).

Compositions containing targeting vectors: The targeting vectors can be lyophilized, mixed with a cryoprotectant, or solubilized or suspended in another diluent (e.g., a buffer or alcohol). The compositions can also include preservatives. Such compositions are within the scope of the present invention and may further include an artificial chromosome (as described further below, including those that contain sequences (e.g., cDNA or genomic sequences) of interest from mammals (e.g., humans, mice or other laboratory animals), other animals (e.g., livestock), plants, or pathogens).

Modified artificial chromosomes: The invention features modified artificial chromosomes, including those produced by the methods described here. The modified artificial chromosomes can include (a) a pair of cleavage sites that flank a packaging/cleavage site of a herpes virus; an ori of a herpes virus; and, optionally, a sequence encoding a first selectable marker and/or a sequence that encodes a detectable marker; (b) a nucleic acid sequence of interest; and (c) a sequence encoding a second selectable marker. Typically, the sequence encoding the first selectable marker is derived from the targeting vector (and is therefore flanked by the cleavage sites) and the sequence encoding the second selectable marker is derived from an unmodified artificial chromosome.

As the modified artificial chromosomes can be generated from the targeting vectors and artificial chromosomes described above, the various elements present in the modified artificial chromosomes can be any of those described above. For example, the cleavage sites can be LoxP elements, FRTs, or unique restriction sites; the selectable marker, when present, can be an antibiotic resistance gene (e.g., a sequence that, upon expression, confers resistance to aminopterin, ampicillin, chloramphenicol, erythromycin, hygromycin, kanamycin, spectinomycin, or tetracycline); the sequence of interest can be a genomic or cDNA sequence from a mammalian genome (e.g., the human genome) or the genome of a pathogen (inter alia); and so forth.

Where the modified artificial chromosome is made by methods other than the “recombineering” methods described herein, it may contain fewer elements than described and, in particular, may lack the cleavage sites. Thus, modified artificial chromosomes of the invention can include (e.g., in addition to only their backbone) a packaging/cleavage site of a herpes virus; an ori of a herpes virus; a nucleic acid sequence of interest; and, optionally, sequence encoding a selectable and/or detectable marker. Here, too, these elements can be any of those described in the present specification. Regardless of the precise manner in which the modified artificial chromosome is made, it can be packaged in any of the herpes virus (e.g., a herpes simplex virus, varicella zoster virus, Epstein-Barr virus, or cytomegalovirus). Methods of packaging modified artificial chromosomes are described further below.

In specific embodiments, a targeting vector and an artificial chromosome can be recombined within a cell. The vector and artificial chromosome can be introduced into the cell by methods known in the art, such as calcium phosphate precipitation or electroporation.

Compositions containing modified artificial chromosomes: Compositions that include a modified artificial chromosome are also within the scope of the present invention. The compositions can include a number of unique chromosomes (i.e., modified artificial chromosomes that vary in the sequence of interest they contain). For example, a composition can contain about 2-10, 10-12, 12-50, 50-100, 100-500, 500-1,000, 1,000-5,000, or 5,000-10,000 or more types of unique modified artificial chromosomes. More specifically, the invention features compositions that include a plurality of modified artificial chromosomes, as described herein. The compositions can be formulated for use in methods in which the chromosomes are packaged within herpes viruses (i.e., with diluents in which the viruses can survive). Where a library of modified artificial chromosomes is used, the nucleic acid sequence of interest within at least one member of the plurality will be different from the nucleic acid sequence of interest within at least one other member of the plurality.

While the compositions of the invention may contain substantially pure populations of modified artificial chromosomes (whether of the same or different types), they may also contain targeting vectors and/or artificial chromosomes, and/or reaction products that have not recombined in a desirable way. For example, the compositions can include a substantially pure population of targeting vectors (whether circular or linear) or a mixture of targeting vectors and artificial chromosomes. The altered herpes viruses that package the modified artificial chromosomes may also be formulated in physiologically acceptable compositions (e.g., compositions that are substantially non-toxic). In addition, and depending upon the intended use, any of the compositions described herein can include one or more diluents (e.g., an excipient or carrier) or other agents typically used in pharmaceutical compositions (e.g., ceramics or other fillers or binding agents).

Altered herpes viruses: In another aspect, the invention features altered herpes viruses that have packaged the modified artificial chromosomes. We may refer to these viruses as particles, and they may package the modified artificial chromosomes described herein. A substantially pure population of the particles can be formulated as compositions, and the particles within the population as well as the manner in which they are formulated, may vary depending upon their intended use (e.g., depending upon whether the particles are intended for use in a screening assay or as therapeutic agents). For example, the compositions may further include one or more diluents (e.g., one or more excipients or carriers) or other pharmaceutical agents.

The altered herpes viruses can infect cells, and a host cell that includes such an altered herpes virus (e.g., a cell ex vivo) is within the scope of the present invention. We may refer to host cells as “permissive” for herpes virus propagation. The host cell can be a mammalian cell (e.g., a human cell), and the cell can be one that is maintained in tissue culture. For example, the host cells can be within an organ, tissue, or cell culture. Varying numbers of cells within the organ, tissue, or cell culture may carry the altered herpes virus (complete or uniform transduction is not required). The host cells can also be arrayed on a substrate, and arrays in which cells at least one of the positions within the array are infected with a different altered herpes virus than are cells at least one other position within the array are also within the scope of the present invention. Regardless of the source of the host cell, it can vary in its developmental stage. For example, mammalian host cells can be embryonic or fetal cells or can be obtained from any age animal (e.g., a young, adolescent, adult, or aged animal).

The altered herpes viruses and cells containing them can also be formulated within compositions (e.g., physiologically acceptable compositions), and such compositions are within the scope of the invention. In one embodiment, the composition can include a plurality of altered herpes viruses, all of which (or substantially all of which) express the same transgene. Alternatively, the composition can include a plurality of altered herpes viruses, and the nucleic acid sequence of interest within the modified artificial chromosome of at least one member of the plurality can be different from the nucleic acid sequence of interest within the modified artificial chromosome contained by at least one other member of the plurality. In some embodiments, very few members of the plurality will contain the same transgene (i.e., the plurality can be extremely heterogeneous).

Methods of generating an altered herpes virus: The methods of the invention include methods of generating a herpes virus that includes a modified artificial chromosome or that can package and express a transgene carried by the chromosome. We may refer to these viruses as altered herpes viruses or as herpes virus particles. The methods can be carried out by (a) providing a cell, which may or may not include a nucleic acid sequence that encodes an accessory protein; (b) transfecting the cell with (i) one or more packaging vectors that, individually or collectively, encode one or more of the herpes virus structural proteins but do not include a functional herpes virus ori and (ii) a modified artificial chromosome; and (c) culturing the cell for a time and under conditions that permit the cell to produce an altered herpes virus. In lieu of steps (a) and (b), one may simply obtain the required cell (i.e., steps (a) and (b) may be collapsed into a single “providing” step). The herpes virus can be any of those types referenced above, and the cell can be any permissive cell (e.g., a mammalian cell (e.g., a human cell)). Although the particular cell type is not limited, one could use a neuron, a fibroblast, a blood cell, a hepatocyte, a keratinocyte, a melanocyte, a glial cell, an endocrine cell, an epithelial cell, a muscle cell, a bone cell, a prostate cell, a testicular cell, or a germ cell. The cell may also be diseased (e.g., malignant) and, as noted above, obtained at any developmental stage or at any stage of differentiation.

Where a sequence encoding an accessory protein is employed, that sequence can also encode a biologically active fragment or mutant of an accessory protein (e.g., a biologically active fragment or other mutant of vhs or VP16. The nucleic acid sequence encoding the accessory protein can include the sequence of vhs or VP16 or a degenerate variant thereof. The vhs protein has an endoribonucleolytic activity that is important in the time-dependent progression of HSV gene expression and virion assembly, and VP16 is a strong transcriptional activator protein. Any of the invention that include expression of a vhs protein can employ, for example, an HSV-1 vhs protein, an HSV-2 vhs protein, an HSV-3 vhs protein, bovine herpes virus 1 vhs protein, bovine herpes virus 1.1 vhs protein, gallid herpes-virus 1 vhs protein, gallid herpes virus 2 virion hsp, suid herpes virus 1 vhs protein, baboon herpes virus 2 vhs protein, pseudorabies vhs protein, cercopithecine herpes virus 7 vhs protein, meleagrid herpes virus 1 vhs protein, equine herpes virus 1 vhs protein, or equine herpes virus vhs protein). Any of these proteins can be operatively coupled to its native transcriptional control element(s) or to an artificial control element (i.e., a control element that does not normally regulate its expression in vivo).

The sequence encoding VP16 or a transcriptional activator that mimics VP16 can be introduced into packaging cells prior to the packaging components. The activation domain can be replaced with another regulatory protein so long as the signal that regulates the CAT/GRATATGARAT sequences is retained. While “pre-loading” the packaging cells with VP16 is not essential, it can be done within the context of the present methods, and it can lead to an additional enhancement of amplicon particle titers. Moreover, the methods can be carried out with cells in which VP16, or a biologically active variant thereof, is stably expressed (methods to achieve stable expression are known in the art). VHS, or a biologically active variant thereof, can also be stably expressed so long as its expression can be suitably controlled. For example, one can control the expression of a sequence encoding VHS (or a biologically active fragment or other mutant thereof) by placing it in the context of a tetracycline, RU46, or ecdysone system. Similarly, the methods in which herpes virus amplicon particles are generated by transfecting a cell with a sequence encoding VHS can be carried out with VHS (e.g., the VHS encoded by gene UL41) or with a mutant VHS, particularly one in which RNAse activity is reduced. Examples of VHS mutations that lead to abolished RNAse activity are the R7, Sc243, and M384 mutations described previously by Jones et al. (J. Virol. 69:4863-4871, 1995).

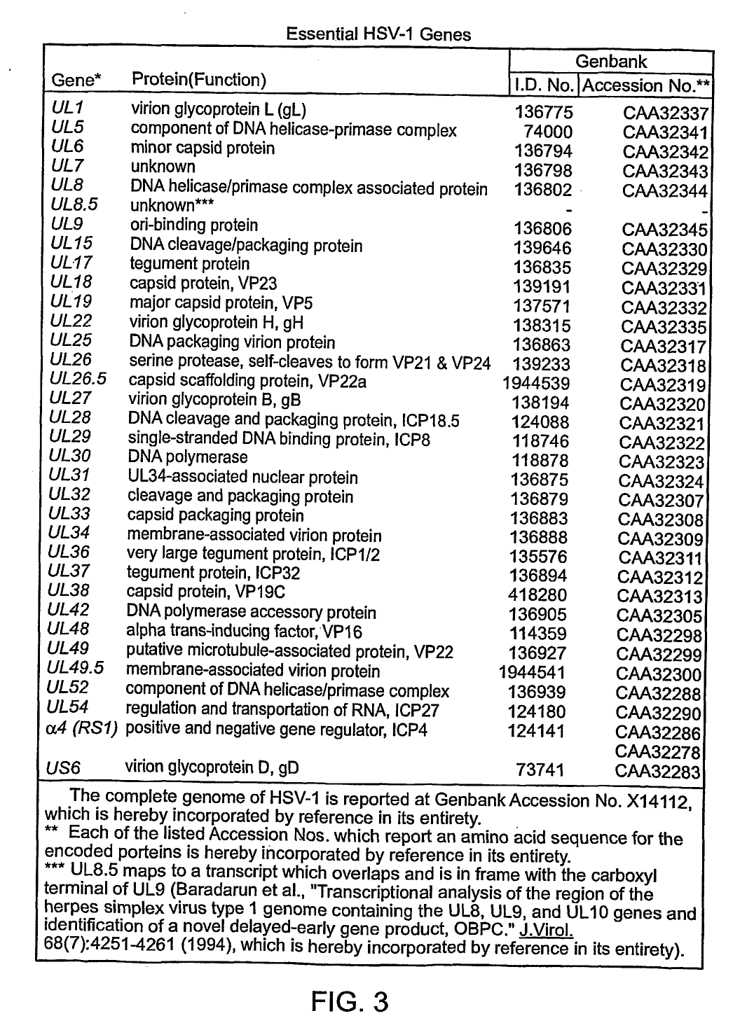

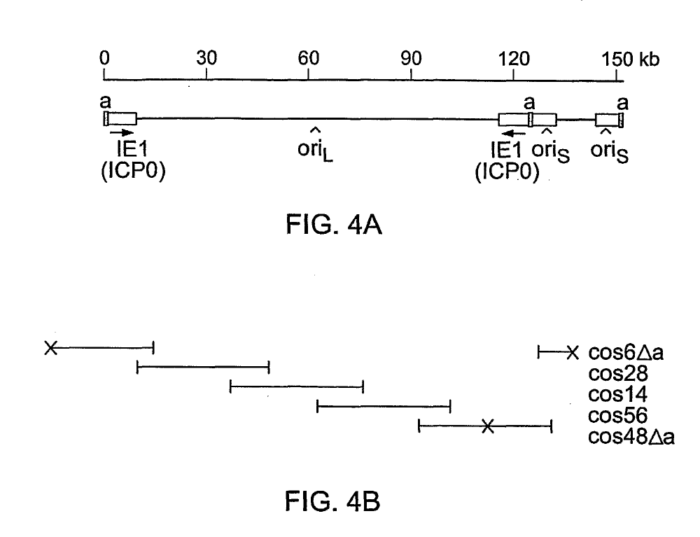

The packaging vectors employed can be a YAC, a BAC, a HAC, an F element plasmid, a cosmid or a set of cosmids. For example, one can use a set of cosmids that, individually or collectively, encode all essential HSV genes but exclude all cleavage/packaging signals. For example, the cosmids can include cos 6Δa, cos 28, cos 14, cos 56, and cos 48Δa (see FIGS. 4A and 4B). Essential HSV-1 genes are listed in the table of FIG. 3.

Methods of producing host cells with stably integrated transgenes: In alternative embodiments, the cell can also be transfected with a sequence encoding an enzyme that catalyzes a reaction within the cell, the consequence of the reaction being that the sequence carried by the modified artificial chromosome (e.g., the transgene) is inserted into the genome of the cell. The enzyme can be, for example, a transposase (e.g., the transposase is encoded by Sleeping Beauty). Although HSV amplicon particles can efficiently infect non-dividing cells and express transgenes therein, long-term expression in actively dividing cells has proven difficult. Combined the Tcl-like Sleeping Beauty (SB) transposon system with the modified artificial chromosomes and packaging vectors described herein can create herpes virus particles that can integrate into the genomes of both dividing and non-dividing cell types. Vector integration within cells can extend the period of expression (e.g., expression of a protein of interest or of a therapeutic agent encoded by a modified artificial chromosome).

Isolated altered herpes viruses and compositions containing same: In subsequent steps, the herpes virus particles can be isolated from the cell or from the medium in which the cell was cultured, and such isolated viruses and compositions (e.g., pharmaceutical compositions) containing them are within the scope of the present invention. The herpes virus particles can be partially purified from the cell or substantially purified (e.g., following a purification process, the herpes virus particles can constitute at least 85% (e.g., 90, 95, 99% or more) of the purified product). The compositions include cell-based and cell-free compositions. For example, the composition can include a host cell transduced with any of the altered herpes viruses described herein. The cell can be a mammalian cell (e.g., a human cell) and, with respect to cell type, can be any somatic cell susceptible to infection (e.g., a neuron or fibroblast). As noted, cells containing modified artificial chromosomes and/or altered herpes viruses that have packaged them can be arrayed, and such cellular arrays are within the scope of the present invention.

Methods of isolating herpes viruses from cells are known in the art and those methods can be applied to isolate the altered herpes viruses described herein. For example, the isolation methods can include lysing particle-containing cells; clearing or reducing the cellular debris; and applying the cleared remainder to a sucrose density gradient (particles come to reside at the interface). Purification can also be achieved by affinity chromatography. For example, one can immobilize an antibody or a fragment thereof (e.g., a single chain antibody that may be humanized) that recognizes a protein on the herpes virion (e.g., an env protein). The antibody can be immobilized on a column or other solid support. Once immobilized, the antibody can be exposed to a sample containing altered herpes viruses under conditions in which the antibody can specifically bind the particles. After the remainder of the sample is washed away, the antibody-virus interaction can be broken (e.g., the complex can be cleaved with a protease (e.g., an endopeptidase, a viral protease, or a combination thereof). Preferably, no protein is cleaved from the altered herpes virus.

Methods of identifying biologically active proteins: Other methods of the invention include methods of determining whether a protein alters the physiology of a cell. The protein can be a full-length or naturally occurring protein or a fragment or other mutant thereof (which may or may not retain biological activity). The methods can be carried out by (a) providing a cell; (b) exposing the cell to a herpes virus that includes a modified artificial chromosome having a sequence that encodes the protein; and (c) determining whether the protein alters the physiology of the cell. The modified artificial chromosome can be a modified artificial chromosome described supra. Preferably, the cell is exposed to the herpes virus for a time and under conditions in which the herpes virus transduces the cell and the nucleic acid sequence once carried by the artificial chromosome (the transgene or sequence of interest) is expressed as a protein within the cell. The cell can be any type of cell injectable by the altered herpes virus. For example, the cell can be a mammalian cell (e.g., a human cell). More specifically, the cell can be a neuron, a fibroblast, a blood cell, a hepatocyte, a keratinocyte, a melanocyte, a glial cell, an endocrine cell, an epithelial cell, a muscle cell, a bone cell, a prostate cell, a testicular cell, or a germ cell. The cell can also be diseased (e.g., malignant) and/or obtained at any developmental stage or at any stage of differentiation.

Determining whether the protein alters the physiology of the cell can be done by determining whether the protein (that encoded by the sequence of interest) alters the rate at which the cell proliferates, the length of time the cell survives, or the number of times the cell divides. Alternatively, one can determine whether the protein alters a property of a channel in a membrane of the cell (e.g., a calcium channel, a chloride channel, a potassium channel, or a sodium channel). Another way to determine whether the protein alters the physiology of the cell is to determine whether the protein alters the composition of the cell. For example, one can determine whether the protein encoded by the sequence of interest alters (i.e., increases or decreases): the amount of a protein in the cell or secreted by the cell; the activity of a protein in the cell or secreted by the cell; or the amount of a fat or carbohydrate in the cell. Either the protein encoded by the sequence of interest within the herpes virus particle or the protein one examines following infection can be an enzyme, co-enzyme, prosthetic group, transcription factor, growth factor, cofactor, a receptor or a subunit thereof, a signaling molecule, an apoptosis inhibitor, an apoptosis promoter, an oncoprotein or oncogenic protein, a DNA replication factor, a structural protein, a neural protein, a heat shock protein, a histone, an immunomodulatory protein (a cytokine (e.g., an interleukin, an interferon, or a chemokine) or costimulatory molecule (e.g., B7 or CD40L), a tumor-specific antigen (e.g., a prostate specific antigen), or a protein (e.g., an antigen) of an infectious agent (a virus, parasite, or bacterium or a prion protein). For example, one can infect a cell with an altered herpes virus that expresses, within the cell, a transcription factor and subsequently determine whether that expression alters the expression or activity of an oncoprotein or tumor-specific antigen. Alternatively, one can infect a cell with an altered herpes virus that expresses, within the cell, a viral protein and subsequently determine whether that expression alters the expression or activity of an apoptosis inhibitor.

In the event the protein is an enzyme, the enzyme can be an oxidoreductase, transferase, hydrolase, lysase, isomerase, ligase, kinase, phosphatase, protease, lipase, nuclease, polymerase, helicase, a lysosomal enzyme, or a hexosaminidase. Alternatively, or in addition, the protein can be an enzyme substrate (e.g., an amyloid precursor protein, a galactomannan substrate, or N-acetyl aspartate).

In the event the protein is a growth factor, the growth factor can be a brain-derived neurotrophic factor (a BDNF), a cilliary neurotrophic factor (a CNTF), an epidermal growth factor (an EGF), a fibroblast growth factor (an FGF), a glial growth factor (a GGF) or glial cell-derived growth factor, an insulin-like growth factor (an IGF), a nerve growth factor (an NGF) or neurotrophin, or a transforming growth factor (a TGF).

In alternative embodiments, the protein can be a hormone (e.g., melatonin, antidiuretic hormone, proopiomelanocortin, luteinizing hormone, follicle stimulating hormone, adrenocorticotropic hormone, growth hormone, prolactin, thyroid stimulating hormone, cortisol, an androgen, an estrogen, insulin, or a pituitary hormone).

Other proteins that can be expressed and/or assessed are neurotransmitters (e.g., epinephrine, norepinephrine, serotonin, acetylcholine, histamine, endorphin, substance P, or dopamine). The protein expressed and/or assessed can also be one that is associated with cancer (e.g., an oncogenic protein such as fos, jun, ras, pRB, or p53). Where the protein is a receptor, it can be a neurotransmitter receptor, a cytokine receptor (e.g., a growth factor receptor) or a hormone receptor.

Methods of identifying therapeutic agents: Other methods of the invention include methods of identifying a candidate therapeutic agent by: (a) providing a cell; (b) exposing the cell to (i) the candidate therapeutic agent and (ii) a herpes virus comprising a modified artificial chromosome having a sequence of interest that encodes a protein; and (c) determining whether the candidate therapeutic agent affects the way in which the protein alters the physiology of the cell. The modified artificial chromosome can be a modified artificial chromosome described supra. Preferably, the cell is exposed to the herpes virus for a time and under conditions in which the herpes virus transduces the cell and a nucleic acid sequence of interest carried by the artificial chromosome is expressed as a protein within the cell. The candidate therapeutic agent can be applied before the cell is exposed to the altered herpes virus, simultaneously with (or in close sequence with) the application of the altered herpes virus, or after the virus has transduced the cell. The candidate therapeutic agent can be essentially any type of therapeutic agent, including a small molecule, a nucleic acid, or a protein (e.g., a protein described herein or an antibody that functions as an agonist or antagonist of a protein described herein), and the modified artificial chromosome can be any of those described herein. Similarly, the nucleic acid sequence of interest can be a genomic sequence or a cDNA sequence (e.g., a genomic human sequence or a human cDNA sequence or a sequence of a pathogen such as a virus, bacterium, fungus, parasite, or prion). Where nucleic acids are tested as therapeutic agents, those nucleic acids can mediate RNAi or may be more traditional antisense oligonucleotides. The nucleic acids can also encode functional proteins. Small molecules can be any organic or inorganic molecule, including those available in compound libraries, many of which are publicly or commercially available.

Methods of delivering therapeutic agents to a patient: Where the therapeutic agent is a protein, the altered herpes viruses described herein can be used to deliver that protein to a cell in vivo or in cell culture. The therapeutic agent can be one that is discovered in the screening methods of the present invention or a protein presently known or suspected of being therapeutic for a given disorder (i.e., the altered herpes viruses of the present invention can be used to deliver previously identified therapeutic proteins). Accordingly, the invention features methods of identifying a therapeutic protein, whether by using a screening method described herein or by surveying information within the public domain, and delivering that therapeutic protein to a cell in vivo or in cell culture. The protein can be delivered by exposing the cell to an altered herpes virus that expresses the protein for a time and under conditions that permit the virus to transducer the cell. In other embodiments, once the therapeutic protein is identified (by, for example, the screening process described above), it can be delivered to a patient by other vehicles. For example, it can be expressed by another viral vector (e.g., a retrovirus) or another type of vector (e.g., a plasmid).

In the event altered herpes viruses are introduced into cells in culture, the host cells can then be administered to patients. The cells administered may have been obtained initially from a patient and subsequently placed in culture; the administration can be of an autologous cell. However, the invention is not so limited. The cell can be any of a wide variety of types, so long as it is permissive for herpes virus propagation and compatible with the patient being treated (i.e., so long as the cell does not induce unacceptable side effects). As noted above, cells can be exposed to an altered herpes virus in combination with a vector that expresses an enzyme (e.g., a transposase) that facilitates chromosomal integration of the transgene carried by the modified artificial chromosome. Such an enzyme can be used when the cells are intended for administration to a patient, and cells and cell-based compositions bearing chromosomally integrated transgenes, originally carried by, for example, an artificial chromosome, are within the scope of the invention. We note, however, that the transgene may also be present episomally within a cell.

Generally, the patient may have any of a wide variety of diseases or conditions. For example, the patient can have an infectious disease. These patients may have been, or may become, infected with a wide variety of agents (including viruses such as a human immunodeficiency virus, human papilloma virus, herpes simplex virus, influenza virus, a pox virus, Ebola virus, bacteria (including eubacteria and archaea), such as Escherichia (e.g., E. coli) a Staphylococcus, Streptococcus, Campylobacter (e.g., C. jejuni), Listeria (e.g., L. monocytogenes), Salmonella, Shigella, or Bacillus (e.g., B. anthracis), a parasite, a mycoplasma, or an unconventional infectious agent such as a prior protein). The patient may also have, or be at risk for developing, a cancer (e.g., a leukemia or lymphoma) or other cellular proliferative disorder (e.g., a benign growth). Patients diagnosed as having a neurological deficit (e.g., a cognitive defect, motor disorder (including paralysis or paresthesis) or a sensory loss (e.g., an impaired sense of hearing, taste, smell, or siglit), or a neurological disease (e.g., Parkinson's disease, Alzheimer's disease, or Huntingtin's disease) are also amenable to treatment. Other patients include those having a disease or condition that results from a genetic defect (e.g., cystic fibrosis) or birth injury (e.g., brain impairment due to oxygen deprivation). A patient “with” a disorder can be a patient diagnosed as having that disorder. Accordingly, a patient can be treated after they have been diagnosed as having a cancer, an infectious disease, or a neurological disorder, etc. . . . Similarly, since certain agents of the present invention can be formulated as vaccines, patients can be treated before they have developed the cancer, infectious disease, neurological disorder, or the like. Thus, “treatment” encompasses prophylactic treatment. For example, patients who have experienced a loss of hearing can be treated at any time, including before the loss occurs (e.g., altered herpes viruses carrying a therapeutic transgene can be administered before the patient is exposed to some agent, such as a chemotherapeutic agent or industrial hazard, that may damage their hearing).

In addition, the assays can be used to identify agents useful in the treatment of lysosomal storage diseases and treatment can occur, for example, by expressing MPS I-VIII, hexoaminidase A/B, or another protein identified by the screening methods. Other target diseases and populations of patients having these diseases include Lesch Nyhan syndrome (treatment can occur, for example, by expressing HPRT or another protein identified by the screening methods), amyloid polyneuropathy (treatment can occur, for example, by expressing B-amyloid converting enzyme (BACE), amyloid antisense sequences, or another sequence or protein identified by the screening methods), Alzheimer's Disease (treatment can occur, for example, by expressing a nerve growth factor such as NGF, ChAT, BACE, etc., or a protein identified by the screening methods), retinoblastoma (treatment can occur by, for example, expressing pRB or a protein identified by the screening methods), Duchenne's muscular dystrophy (treatment can occur by expressing Dystrophin or a protein identified by the screening methods), Parkinson's Disease (treatment can occur, for example, by expressing GDNF, Bcl-2, TH, AADC, VMAT, sequences antisense to mutant alpha-synuclein, or a protein identified by the screening methods), Diffuse Lewy Body disease (treatment can occur, for example, by expressing a heat shock protein, parkin, or antisense or RNAi molecules to alpha-synuclein, or a protein or inhibitory sequence identified by the screening methods), stroke (treatment can occur by, for example, expressing Bcl-2, HIF-DN, BMP7, GDNF, or other growth factors or TPA, or by expressing a protein identified by the screening methods), a tumor, such as a brain tumor (treatment can occur by, for example, expressing angiostatin, antisense VEGF, antisense or ribozyme to EGF or scatter factor, or pro-apoptotic proteins or a protein identified by the screening methods), epilepsy (treatment can occur by, for example, expressing GAD65, GAD67, or pro10 apoptotic proteins or a protein identified by the screening methods), or arteriovascular malformation (treatment can occur by expressing proapoptotic proteins or a protein identified by the screening methods).

In all instances where a full-length protein can be used in the methods of the invention, a biologically active fragment or other mutant thereof can also be used. It follows that nucleic acid sequences that encode such biologically active fragments or mutants (e.g., proteins that are mutant by virtue of including one or more amino acid substitutions or additions) can also be used. These nucleic acid and protein variants can be used in methods for making a composition described herein (e.g., a modified artificial chromosome or altered herpes virus); in methods for screening for therapeutic agents; in methods for making pharmaceutical compositions; or in methods for administering the agents or compositions.

Kits: Kits that can be used to generate modified artificial chromosomes and/or altered herpes viruses as well as kits that can be used to screen for drug targets and therapeutic agents are also within the scope of the present invention. For example, the invention features a kit that includes a targeting vector described herein and, optionally, an artificial chromosome that contains a nucleic acid sequence of interest. The kits can also contain a composition (e.g., a physiologically acceptable composition) that contains such chromosomes or viruses. Alternatively, or in addition, the kits can contain host cells (e.g., prokaryotic host cells that include, or can include, a modified artificial chromosome or eukaryotic cells that include an altered herpes virus). Other kits can include one or more of the components useful in generating modified artificial chromosomes or altered herpes viruses. For example, a kit can include an enzyme to facilitate recombineering, a host cell, a helper virus, and/or a modified artificial chromosome. Alternatively, or in addition, the kits may include an enzyme, or a vector that encodes an enzyme, that mediates integration of the transgene carried by the modified artificial chromosome into the genome of a host cell. Where the kits are intended to aid screening assays, they may include cellular arrays and reagents for assessing physiological function. For example, the kits can include one or more reagents to assess the effect of a transgene on a cellular process (e.g., cell survival, the rate of cell division, differentiation potential, or regenerative activity). Any of the kits can also include instructions for use. The instructions can be conveyed by a variety of media (e.g., print, audiotape, videotape, CD, DVD, and the like). The compositions of the kits of the invention are preferably packaged in sterile form.

### EXAMPLES

### Example 1

In this recombineering protocol, the vector to be modified (a BAC) was placed into E. coli strain EL250 containing defective lambda prophage.

To prepare competent cells, we streaked E. coli strain EL250 cells from a glycerol stock stored at −80° C. onto an LB plate. From an individual colony that arose on the plate, we inoculated a 50 ml LB culture (250 ml Ehrlenmyer flask) and grew the liquid culture at 32° C. overnight in a shaking incubator (2000 rpm). We removed the culture and chilled the flask in a slurry of ice and water, gently shaking by hand to chill the cells quickly. We transferred 10 ml of culture to a 15 ml conical tube and centrifuged it at 3500 rpm in a Beckman SLA-1500 rotor for 5 min at 4° C. (stopping with no brake). After pouring off the supernatant, we gently resuspended the cells in 10 ml of sterile double distilled water (ddH₂O). The resuspended cells were centrifuged at 3500 rpm in a Beckman SLA-1500 rotor for 5 minutes at 4° C. (and allowed to slow with no brake). The supernatant was again removed and the cells were resuspended in 1 ml of sterile ddH₂O. The washing step was repeated twice more, for a total of three washes with 1 ml of sterile ddH₂O. After a final centrifuge, we resuspended the cells in 80 μl of sterile ddH₂O to obtain cells resuspended in a final volume of ˜100 μl.

To introduce the targeting vector, we combined 10-100 ng of vector DNA with 50 μl of competent cells in a 1.5 ml microfuge tube and chilled the mixture on ice for 5 minutes. We then pipetted the cells into a prechilled 1 mm cuvette (BioRad) and electroporated them with 1.75 kV and 186 Ohms. We added 450 μl of SOC media to the cuvette and transferred the entire contents to 1.5 ml microfuge tube, which was incubated at 32° C. for 30 minutes. We then plated the cells using selective media (Ram^{r} for BAC).

To prepare a linear targeting vector, a selected circular targeting vector can be digested and the products separated by gel electrophoresis. The desired fragment can then be cut out of the gel and purified. Alternatively, a linear nucleic acid to be used for recombineering can be generated by PCR. The desired product can then be isolated from a gel (e.g., an acrylamide gel).

The RED genes used for recombination are under the control of a heat inducible promoter. The strains are briefly heated to 42° C. to allow expression and then chilled to reduce activity until the introduction of the PCR cassette through electroporation.

To induce RED genes, we inoculated 5 ml of LB medium containing a selective antibiotic with strain EL250 cells containing the vector to be modified. The cells were grown overnight at 32° C., and one ml of the overnight culture was inoculated into 50 ml of fresh media (in a 500 ml flask). The culture was grown at 32° C. until the OD₆₀₀ equaled 0.5-0.8. We then transferred 10 ml of culture to a 125 ml flask and placed it in a 42° C. waterbath for 15 minutes. The flask was then moved to a slurry of ice and water and swirled gently to quickly chill the cells. We then transferred the culture to a 15 ml conical tube and centrifuged it at 3500 rpm in a Beckman SLA-1500 rotor for 8 min at 4° C. (the centrifuge slowed with no brake). We poured off the supernatant and gently resuspended the cells in 1 ml of sterile ddH₂O. The cells were pelleted again in a 4° C. microfuge at maximum speed for 20 seconds. We repeated the washing step twice more, for a total of three washes in 1 ml of sterile ddH₂O. After the final spin, we resuspended the cells in 100 μl of sterile ddH₂O.

For the final electroporation, we placed 50-100 ng of a PCR-generated cassette and 50 μl of competent cells in a 1.5 ml microfuge tube and chilled the tube on ice for 5 minutes. We pipetted the cells into a prechilled 1 mm cuvette (BioRad) and electroporated them using 1.75 kV and 186 Ohms. Following electroporation, we added 950 μl of SOC media to the cuvette and transferred the entire contents to a 1.5 ml microfuge tube, which we incubated at 32° C. for 30 minutes. The cells were then plated on selective medium (Ram^{r} plates for BAC).

### Example 2

This study employs amplicon BAC engineering to discover new molecules involved in neural regeneration and repair.

A major obstacle in the treatment of traumatic injuries to the brain or spinal cord is the incapacity of neurons in the adult central nervous system (CNS) to regenerate damaged axons. One important factor attributed to this regenerative failure is the growth inhibitory environment encountered by injured axons. It is well established that adult CNS neurons possess the intrinsic machinery to grow axons, and when provided with a favorable environment, may extend axons over long distances. Multiple lines of evidence point to adult CNS myelin as a major barrier for axonal growth and regeneration. Several myelin-derived inhibitors have been identified, including myelin associated-glycoprotein (MAG), Nogo-A, oligodendrocyte-myelin glycoprotein (OMgp) and most recently, Semaphorin 4D. In addition, chondroitin sulfate proteoglycans and secreted semaphorins associated with the glial scar contribute to the growth inhibitor environment of injured CNS tissue (Filbin, Nature Rev. Neurosci. 4:703-713, 2003).

The recent identification of a neuronal surface receptor for Nogo66, called NgR1 (former NgR), provides for the first time mechanistic insights into Nogo function. NgR1, a member of a leucine-rich repeat (LRR) family, is linked to the cell surface through a glycosylphosphatidyl inositol (GPI) anchor and forms a heteromeric complex with p75NTR and LINGO-1 to signal inhibition across the neuronal cell membrane. Although Nogo, MAG and OMgp lack sequence homologies, they all bind to the NgR1 and recent data suggest that the myelin inhibitory proteins Nogo, MAG, and OMgp all signal growth inhibition through a NgR1/p75NTR/LINGO-1 receptor complex (Mi et al., Nature Neurosci. 7:221-228, 2004).

Considerable progress has been made in identifying myelin inhibitory proteins and their receptors. While growing evidence suggests that RhoA is a key mediator of growth inhibition, the molecular events leading to growth cone collapse and a net loss of actin polymerization at the leading edge of an (injured) axon are not well defined. Recent work suggests that conventional isoforms of PKC are upstream of RhoA and that neuronal expression of dominant negative forms of conventional PKCs attenuates myelin inhibition (Sivasankaran et al., Nature Neurosci. 7:261-268, 2004).

Perhaps most interestingly from a therapeutic point of view are recent findings showing that adult mammalian neurons can be “primed” by pre-exposure to neurotrophins (BDNF or NGF). Priming leads to a transcription/translation-dependent silencing of the classical RhoA inhibitory signaling pathway, which allows adult neurons to extend processes in the presence of myelin inhibitory proteins. Priming is mediated by activation of the cAMP-PKA pathway, which leads to CREB-mediated gene expression. One of the down-stream products of priming has been identified as arginase-1 (Cai et al., Neuron 35:711-719, 2002). Consistent with a key role in ‘primed neurons’, ectopic expression of arginase-1 allows neurons to grow process extensions on a MAG/myelin substrate.

Here, we propose to use an in vitro neurite outgrowth assay on myelin substrate combined with HSV-vector mediated gene transfer to screen for gene products that attenuate or overcome myelin-mediated inhibition of neurite outgrowth. As a control experiment, we propose to introduce arginase-1 into postnatal cerebellar granule neurons (CGNs) using HSV-mediated gene transfer. Neurite length of arginase-1 expressing CGCs will be quantified and compared to CGCs infected with a control HSV-vector carrying a reporter transgene.

Arginase-1 positive control: As a positive control for the proposed screen, HSV-BAC mediated neuronal expression of arginase-1, an enzyme previously shown to allow neurons to grow in the presence of myelin inhibitory proteins, will be used to demonstrate the feasibility of our approach.

In a first series of experiments, we will demonstrate expression of arginase-1 from cells infected with an HSV vector carrying a retrofitted arginase-1 BAC(HSV-BAC/arginase-1; a modified artificial chromosome). Expression will be monitored by Western blotting of HSV-BAC/arginase-1 infected COS-7 cells and immunocytochemistry of neurons infected with HSV-BAC/arginase-1. BAC clones carrying the arginase-1 gene will be ordered from BACPAC and retrofitted with HSV amplicon sequences (for details see below). For immunoblotting and inimunocytochemistry, we will use a polyclonal anti-arginase-1 antibody (Abcam ab211). To confirm that HSV-BAC/arginase-1 infected neurons overexpress arginase-1, we will double stain for arginase-1 and the neuron-specific marker TuJ1 using the anti-class III tubulin antibody (TuJ1; Promega).

Next, we will address whether HSV-BAC/arginase-1 mediated overexpression of arginase-1 in DRG neurons overcomes myelin mediated inhibition of neurite outgrowth. This will allow us to calibrate the neurite outgrowth assay (i.e., to establish myelin concentrations that allow for a large shift in neurite length following overexpression of arginase-1).

Next, we will determine the dilution of HSV-BAC/arginase-1 that still leads to a significant change in neurite length in our functional assay. Serial dilutions of HSV-BAC/arginase-1 with a control HSV-lacZ vector will be used to infect primary neurons. This will allow us to determine the complexity of viral pools optimal for the proposed screen and give an estimate of how many viral pools will have to be screened to cover the entire genome at least twice.

To show that we can identify arginase-1 from a complex viral pool containing HSV-BAC/arginase-1, the original pool will be divided into sub-pools and tested in our functional assay until single HSV clones are obtained. The BAC DNA of the identified HSV will be sequenced directly, or subcloned to demonstrate it contains the arginase-1 gene.

Preparation of Hsv-Bac amplicon library: the Herpes Simplex Virus (Hsv) amplicon vector has proven useful for highly efficient gene transfer into many mammalian cell types. As noted above, the amplicon is a circular DNA requiring only two cis elements from a herpes virus for production in virions. These are the “a” sequence, which is required for packaging, and an HSV origin (ori) of replication. These two sequences are sufficient to confer onto a DNA plasmid the ability to be replicated, cleaved, and inserted into an HSV viral envelope. By transducing cells with amplicons and amplicon-associated vectors, we are able to measure functional outcomes associated with the expression of transferred genes such as myelin responsiveness of primary neurons.

We will use a herpes amplicon library carrying murine BACs to identify genes important for CNS regeneration and repair. We propose to construct a library of HSV-BACs each containing a unique segment of chromosomal DNA from a human. Specifically, we propose to use BAC engineering techniques to generate this library. These HSV-BACs will be packaged into amplicon virions and used, for example, for functional genomic studies.

Generation of HSV-BAC amplicon vector: A BAC will be selected. In making the selection, we may consider its suitability for library construction, which is improved where primer sites for subsequent sequencing are included and backbone sequences divergent from HSV BAC are used in packaging to reduce the risk of recombination. Next, a cassette containing the HSV origins(s) and packaging site and selectable markers (Kan^{r} and dsRED) will be inserted into the BAC using recombineering within several sites of the backbone. Each vector will be tested to determine which construct results in the highest titer of infectious particles.

Construction of HSV-BAC amplicon library (a library of modified artificial chromosomes): We intend to outsource the construction of a human BAC library. We expect a service provider to provide ˜3 times the coverage of the human genome, resulting in ˜9,000 clones with insert size of ˜100 kb. We will ask that these clones be arrayed as single clones on microtiter plates and combined to make pools and super pools.

Packaging of the BAC amplicon library: Our group as well as several others have described helper virus-free packaging methods. We can convert an amplicon DNA, in this case our retrofitted BAC library (containing modified artificial chromosomes), into virus by the co-transfection of a separate BAC carrying the HSV replication and packaging sequences. Utilized in this way, we will prepare a population of virions that should represent, in a one-step packaging process, the complete collection of genomic BAC sequences. These will be characterized in a variety of different assays to make certain that there has been no significant skewing of the population and they will be utilized in cell culture studies to make sure that they are fully effective and capable of transduction.

Identification of Proteins Affecting Growth of Neuronal Processes: To visualize the axon growth inhibitory activity of CNS myelin, a number of robust and well-established cell culture assays may be used. Typically, crude or partially purified myelin fractions are spotted on polylysine and used as a substrate to culture dissociated neurons. A variation of this assay used in our laboratory is to adsorb cryosections of brain and/or spinal cord tissue directly onto glass coverslips in multiwell culture plates. Postnatal neurons are then plated on tissue sections to assess fiber length and number on CNS white matter and gray matter substrates. A major strength of this assay is that it allows us to directly compare fiber growth on gray (permissive) and white (non-permissive) matter. Furthermore, the concentration of tissue-associated inhibitors is likely to be comparable to that encountered by regenerating CNS fibers. This is particularly relevant when dealing with strategies designed to overcome myelin inhibition and promote axonal regeneration in a CNS environment.

Preparations of myelin inhibitory substrate: Myelin inhibitory proteins will be isolated from adult rat spinal cord. Briefly, spinal cords (10 g) from adult rat will be dissected, homogenized, and extracted in ice-cold CHAPS buffer (60 mM CHAPS, 100 mM Tris pH 8.0, 10 mM EDTA, 2% protease inhibitor cocktail (Sigma)). Extracted proteins will be separated from cell debris by two high speed spins (Beckman table top ultracentrifuge; 200,000×g 1 hour each). The clear supernatant will be fractionated over a mono-Q ion exchange column using a BioRad (DuoFlow) FPLC using a linear 0-1M NaCl gradient. Fractions eluting between 0.25-0.5 M NaCl will be pooled, dialyzed and used as an inhibitory substrate for neurite outgrowth.

Primary neuronal cultures: Standard procedures will be used for primary neuronal cultures. For neurite outgrowth assays we use routinely rat P7-P10 cerebellar granule cells and adult rat DRGs.

What is claimed is:

1. A targeting vector comprising a pair of cleavage sites that flank
(a) a packaging/cleavage site of a herpes virus;
(b) an origin of replication (ori) of a herpes virus;
(c) an antibiotic resistance gene; and, optionally
(d) a sequence that encodes a detectable marker.

2. The targeting vector of claim 1, wherein the pair of cleavage sites comprises a pair of LoxP elements or a pair of Flp recombination targets (FRTs).

3. The targeting vector of claim 1, wherein:
when present, each member of the pair of loxP elements comprises the sequence 5′-ataacttcgtataatgtatgctatacgaagttat-3′ (SEQ ID NO: ______) or a biologically active fragment or mutant thereof; and
when present, each member of the pair of FRTs comprises the sequence 5′-gaagttcctattctctagaaagtataggaacttc-3′ (SEQ ID NO: ______) or a biologically active fragment or mutant thereof.

4. The targeting vector of claim 1, comprising a biologically active fragment or mutant of an ori.

5. The targeting vector of claim 1, wherein the herpes virus is a herpes simplex virus, varicella zoster virus, Epstein-Barr virus, or cytomegalovirus.

6. The targeting vector of claim 5, wherein the herpes simplex virus is a type 1 (HSV-1) or type 2 (HSV-2) herpes simplex virus.

7. The targeting vector of claim 5, wherein the herpes simplex virus is a type 3 (HSV-3), type 4 (HSV-4), type 5 (HSV-5), type 6 (HSV-6), type 7 (HSV-7), or type 8 (HSV-8) herpes simplex virus.

8. The targeting vector of claim 1, wherein the herpes virus is a human herpes virus.

9. The targeting vector of claim 1, wherein the detectable marker is a fluorescent protein.

10. The targeting vector of claim 9, wherein the fluorescent protein is a green, red, or blue fluorescent protein.

11. The targeting vector of claim 10, wherein the green fluorescent protein is enhanced green fluorescent protein (EGFP).

12. The targeting vector of claim 10, wherein the red fluorescent protein is DsRed II.

13. The targeting vector of claim 1, wherein the antibiotic resistance gene is a kanamycin resistance gene.

14. The targeting vector of claim 1, wherein the antibiotic resistance gene is a(n) aminopterin, ampicillin, chloramphenicol, erythromycin, hygromycin, spectinomycin, or tetracycline resistance gene.

15. The targeting vector of claim 1, further comprising a second antibiotic resistance gene.

16. A targeting vector comprising:
(a) a packaging/cleavage site of a herpes virus;
(b) an origin of replication (ori) of a herpes virus;
(c) an antibiotic resistance gene; and, optionally
(d) a sequence that encodes a detectable marker, wherein the packaging/cleavage site is flanked by a first pair of cleavage sites and the antibiotic resistance gene is flanked by a second pair of cleavage sites.

17. The targeting vector of claim 16, wherein the first pair of cleavage sites or the second pair of cleavage sites is a pair of LoxP elements or a pair of FRTs.

18. A modified artificial chromosome comprising
(a) a pair of cleavage sites that flank
(i) a packaging/cleavage site of a herpes virus;
(ii) an ori of a herpes virus;
(iii) a first antibiotic resistance gene; and, optionally
(iv) a sequence that encodes a detectable marker;
(b) a nucleic acid sequence of interest, and
(c) a second antibiotic resistance gene.

19. The modified artificial chromosome of claim 18, wherein the artificial chromosome is a yeast artificial chromosome (YAC), a bacterial artificial chromosome (BAC), or human artificial chromosome (HAC).

20. The modified artificial chromosome of claim 18, wherein the pair of cleavages sites comprises a pair of LoxP elements or a pair of FRTs.

21-106. (canceled)
